(19) United States
(12) Patent Application Publication
(10) Pub. No.: US 2020/0062951 A1
(43) Pub. Date: Feb. 27, 2020

(54) THERMALLY ACTIVE COMPOSITE POLYMERIC COMPOSITIONS

(71) Applicant: Wake Forest University, Winston-Salem, NC (US)

(72) Inventor: Nicole Hope LEVI, Winston-Salem, NC (US)

(21) Appl. No.: 16/609,007

(22) PCT Filed: Apr. 30, 2018
(86) PCT No.: PCT/US2018/030146

§ 371 Date: Oct. 28, 2019

Related U.S. Application Data
(60) Provisional application No. 62/491,500, filed on Apr. 28, 2017.

(51) Int. Cl. C08L 65/00; C09D 5/16; C08G 61/12; C09D 7/40; C09D 7/65; A61L 2/232; A61L 2/04; A61L 2/08

(52) U.S. Cl. CPC C08L 65/00; C08G 2261/3247; C08G 61/126; C09D 7/68; C09D 7/67; C09D 7/65; A61L 2/232; A61L 2/04; A61L 2/085; C08G 2261/18; C08L 2201/10; C08L 2207/04; C08G 2261/124; C08G 2261/1412; C08G 2261/228; C08G 2261/3243; C09D 5/165

(57) ABSTRACT

In one aspect, composite polymeric compositions are described herein which, in some embodiments, are thermally responsive to electromagnetic radiation for the destruction of microbes and associated biofilms. Briefly, a composite polymeric composition comprises a polymeric matrix and photo-thermal particles comprising conjugated polymer dispersed in the polymeric matrix, the photo-thermal particles providing sufficient heat to destroy microbial species upon irradiation with infrared radiation.

### RELATED APPLICATION DATA

The present application claims priority pursuant to 35 U.S.C. § 119(e) to U.S. Provisional Patent Application Ser. No. 62/491,500 filed Apr. 28, 2017 which is hereby incorporated by reference in its entirety.

### FIELD

The present invention relates to composite polymeric compositions and, in particular, to composite polymeric compositions exhibiting thermal activity upon exposure to electromagnetic radiation.

### BACKGROUND

Microbial species exist everywhere in the environment. Many microbial species are benign and play critical roles in the proper functioning of various ecosystems. However, a number of microbial species can induce serious or life threatening diseases in humans and animals Surfaces in health treatment facilities, such as hospitals and nursing homes, are extremely susceptible to accumulation of harmful microbial species and associated biofilms. Surfaces of medical diagnostic equipment are also often in danger of microbial and/or biofilm accumulation. Accordingly, these surfaces are routinely cleaned with one or more antimicrobial compositions. In many cases, the cleaning is incomplete and microbial species regenerate quickly, risking the spread of disease. In some instances, heat treatments are employed to effectively kill microbial species. While effective, heat treatments have limited application to large surfaces and equipment comprising sensitive components.

### SUMMARY

In one aspect, composite polymeric compositions are described herein which, in some embodiments, are thermally responsive to electromagnetic radiation for the destruction of microbes and/or associated biofilms Briefly, a composite polymeric composition comprises a polymeric matrix and photo-thermal particles comprising conjugated polymer dispersed in the polymeric matrix. In some embodiments, the photo-thermal particles are nanoparticles, microparticles or mixtures thereof.

In another aspect, articles employing the composite polymeric compositions are provided. In some embodiments, an article comprises one or more surfaces and a coating over the one or more surfaces, the coating comprising a polymeric matrix and photo-thermal nanoparticles comprising conjugated polymer dispersed in the polymeric matrix. Any article not inconsistent with the objectives of the present invention can have surfaces coated with the composite polymeric composition. As described further herein, articles supporting high concentrations of microbes and associated biofilms can be coated with composite polymeric compositions described herein. Various hospital surfaces, medical devices and/or implants can be coated with composite polymeric compositions described herein. In other embodiments, articles can be formed of composite polymeric compositions described herein. In such embodiments, the article can be employed as a heat source for enhancing uptake and/or efficacy of various pharmaceutical compositions, including antimicrobial or antibacterial compositions and/or chemotherapeutic compositions. The article can also exhibit antimicrobial properties in the presence or absence of applied electromagnetic radiation.

In a further aspect, methods of cleaning surfaces are provided. A method of cleaning a surface comprises heating the surface via exposure to electromagnetic radiation to kill and/or disperse microbes, the surface comprising a composite polymeric composition including a polymeric matrix and photo-thermal nanoparticles comprising conjugated polymer dispersed in the polymeric matrix.

These and other embodiments are described in greater detail in the detailed description which follows.

### BRIEF DESCRIPTION OF THE DRAWINGS

FIG. 1 illustrates monomeric species of conjugated polymer according to some embodiments described herein.

FIG. 2 illustrates monomeric species of conjugated polymer according to some embodiments described herein.

FIG. 3 illustrates absorption spectra red-shifting of PCPDTBSe with increasing number average molecular weight according to one embodiment described herein.

FIG. 4 illustrates a synthetic pathway for PCPDTBSE according to some embodiments.

FIG. 5 illustrates formation of a composite polymeric composition according to some embodiments.

FIG. 6 illustrates water heating profiles of composite polymeric compositions when exposed to laser radiation according to some embodiments.

FIG. 7 illustrates ablation of bacterial species by composite polymeric compositions upon exposure to laser radiation according to some embodiments.

FIG. 8 illustrates bacterial biofilm destruction in response to laser irradiated composite polymeric compositions according to some embodiments.

FIG. 9 illustrates antimicrobial properties of composite polymeric compositions in the absence of laser radiation according to some embodiments.

FIG. 10 illustrates bacterial biofilm destruction in response to irradiated composite polymeric compositions according to some embodiments.

FIG. 11 illustrates antimicrobial activity of polymeric composites in conjunction with an antibacterial compound according to some embodiments.

FIG. 12 illustrates antimicrobial activity of polymeric composites in conjunction with an antibacterial compound according to some embodiments.

FIG. 13 illustrates light absorption profiles of composite polymeric coatings according to some embodiments.

FIG. 14 illustrates water heating profiles of articles comprising composite polymeric coatings according to some embodiments.

FIG. 15 illustrates water heating profiles of articles comprising composite polymeric coatings according to some embodiments.

FIG. 16 illustrates water heating profiles of articles comprising composite polymeric coatings according to some embodiments.

FIG. 17 illustrates detergent heating profiles of articles comprising composite polymeric coatings according to some embodiments.

### DETAILED DESCRIPTION

Embodiments described herein can be understood more readily by reference to the following detailed description and examples and their previous and following descriptions. Elements, apparatus and methods described herein, however, are not limited to the specific embodiments present in the detailed description and examples. It should be recognized that these embodiments are merely illustrative of principles of the present invention. Numerous modifications and adaptations will be readily apparent to those of skill in the art without departing from the spirit and scope of the invention.

In one aspect, composite polymeric compositions are described herein which, in some embodiments, are thermally responsive to electromagnetic radiation for the destruction of microbes and/or associated biofilms. In some embodiments, a composite polymeric composition comprises a polymeric matrix and photo-thermal particles comprising conjugated polymer dispersed in the polymeric matrix.

Photo-thermal particles are operable to provide thermal energy to the environment surrounding the polymeric composite when irradiated with radiation falling within the absorption profile of the conjugated polymer. Various conjugated polymeric species exhibiting a thermal response to radiation may be employed in the photo-thermal nanoparticles. For example, conjugated polymeric species having a bandgap ranging from about 1.1 eV to about 1.8 eV can be used in the photo-thermal particles. Bandgap and associated radiation absorption profile of the conjugated polymer can be tailored to spectral requirement(s) of a particular application. As discussed further herein, photo-thermal particles comprising conjugated polymer having an absorption profile in the near infrared region (NIR) can be used for treatment applications where radiation is required to penetrate tissue to reach the nanoparticles. For example, polymeric species of the photo-thermal particles can have an absorption profile of 700 nm to 1200 nm. Further, the spectral response of the photo-thermal particles can be broadened or narrowed by the use of multiple polymeric species or a single polymeric species respectively. In some embodiments, composite polymeric compositions described herein are not damaged or destroyed by the heat generated by the photo-thermal particles in response to radiation. This permits reuse of the polymeric composites over a number of heating cycles.

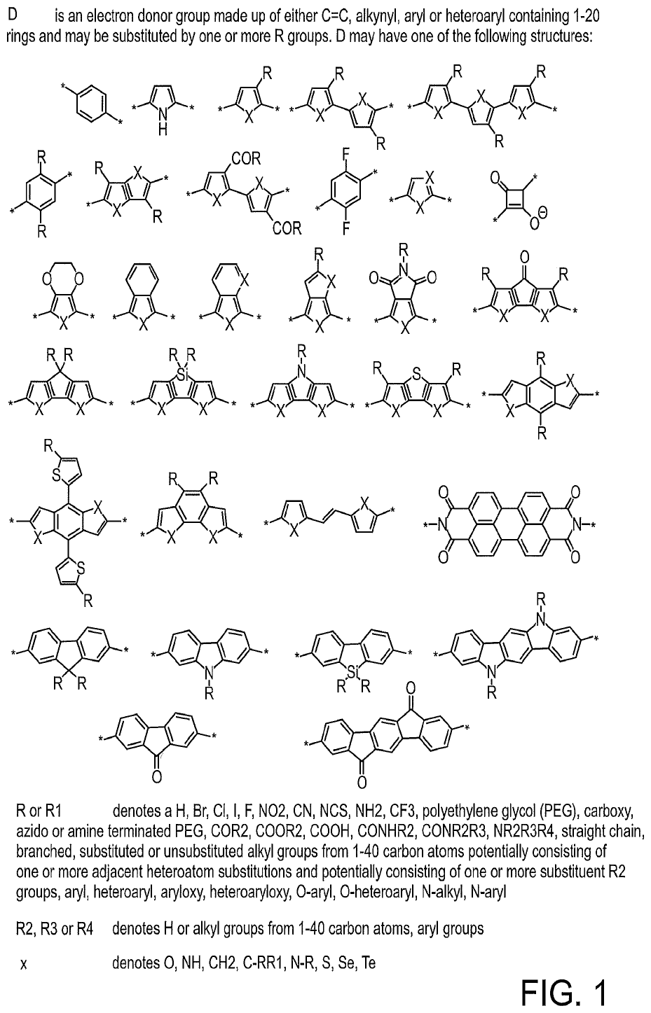

Any thermally responsive conjugated polymer not inconsistent with the objectives of the present invention can be used in the photo-thermal particles. In some embodiments, the conjugated polymer is a homopolymer. For example, a homopolymer can be constructed of a donor monomeric species (D), wherein D is a monocyclic, bicyclic or polycyclic arylene or monocyclic, bicyclic or polycyclic heteroarylene. The arylene structures, in some embodiments, can be fused or linked. A conjugated homopolymer, in some embodiments, is constructed of a monomer selected from the group consisting of aniline, pyrrole, thiophene, 3-substituted thiophene, bithiophene, terthiophene, selenophene, 3-substituted selenophene, isothianaphthene, p-phenylenevinylene, ethylenedioxythiophene, propylenedioxythiophene, 2,7-fluorene, substituted 2,7-fluorene, 2,7-carbazole, substituted 2,7-carbazole, thieno[3,2-b]thiophene, thieno[3,4-b]thiophene, dithienothiophene, cyclopenta[2,1-b:3,4-b′]dithiophene, substituted cyclopenta[2,1-b;3,4-b′]dithiophene, dithieno[3,2-b:2′,3′-d]silole, benzo[1,2-b;4,5-b′]dithiophene, benzo[1,2-b;3,4-b]dithiophene, indolo[3,2-b]carbazoles, dithieno[3,2-b:2′,3′-d]pyrrole, diketopyrrolopyrrole, pentacene, heptacene and perylenediimine. Some suitable donor monomeric species are further illustrated in FIG. 1. In the structures of FIG. 1, X can be O, N, S or Se. In some embodiments comprising more than one X, each X can independently be O, N, S, Se or Te. In addition, R, R₁, R₂ and R₃ can independently be selected from the group consisting of hydrogen, alkyl, alkenyl, aryl, heteroaryl, O-alkyl, O-alkenyl, and O-aryl. An alkyl, alkenyl, aryl, heteroaryl, O-alkyl, O-alkenyl, or O-aryl group, in some embodiments, comprises between 1 and 30 carbon atoms or between 1 and 15 carbon atoms.

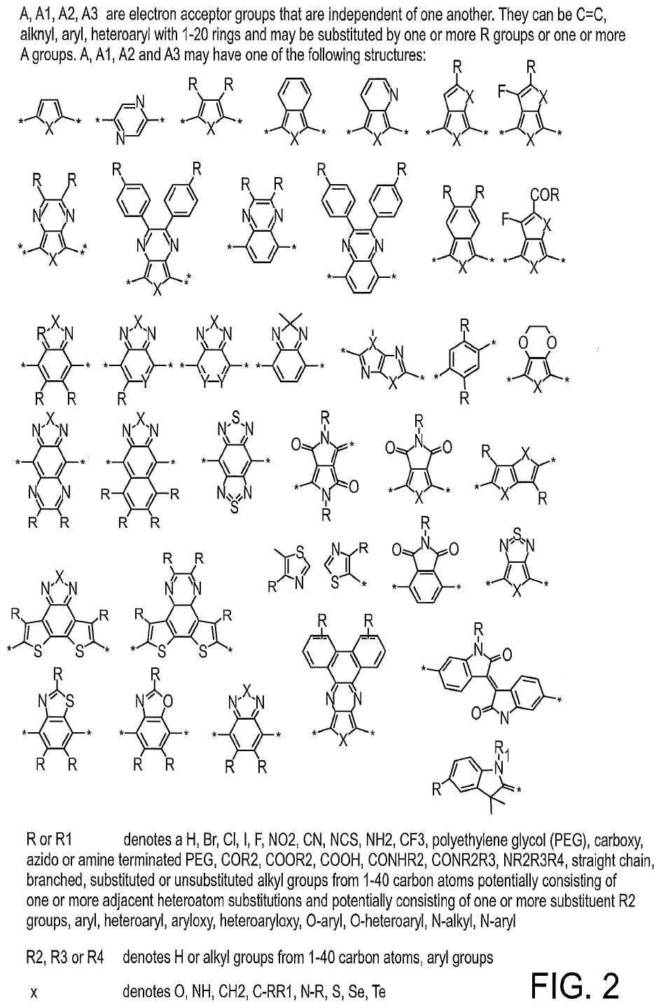

Additionally, a conjugated homopolymer of the photo-thermal particles can be constructed of an acceptor monomeric species (A, A₁, A₂, A₃ in formula herein), wherein the acceptor monomeric species is a monocyclic, bicyclic or polycyclic arylene or monocyclic, bicyclic or polycyclic heteroarylene. The arylene structures, in some embodiments, can be fused or linked A conjugated homopolymer, in some embodiments, is constructed of a monomer selected from the group consisting of pyrrole, aniline, thiophene, ethlyenedioxythiophene, p-phenylenevinylene, benzothiadiazole, pydridinethiadiazole, pyridineselenadiazole, benzoxadiazole, benzoselenadiazole, thieno[3,4-b]pyrazine, thieno[3,4-b]thiophene, thieno[3,2-b]thiophene, [1,2,5]thiadiazolo[3,4-g]quinoxaline, pyrazino[2,3-g]quinoxaline, thienopyrrolidinone and isothianaphthene. Some suitable acceptor monomeric species (A, A₁, A₂, A₃) are further illustrated in FIG. 2. In the structures of FIG. 2, X can be O, N, S, Se or Te. In some embodiments comprising more than one X, each X can independently be O, N, S or Se. In addition, R, R¹ and R² can independently be selected from the group consisting of hydrogen, alkyl, alkenyl, aryl, heteroaryl, O-alkyl, O-alkenyl, and O-aryl. An alkyl, alkenyl, aryl, heteroaryl, O-alkyl, O-alkenyl, or O-aryl group, in some embodiments, comprises between 1 and 30 carbon atoms or between 1 and 15 carbon atoms. In some embodiments, an acceptor monomeric species is a diketopyrrolopyrrole. For example, a diketopyrrolopyrrole is of the formula:

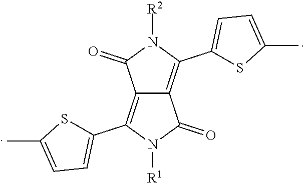

wherein R¹ and R² are defined above.

Alternatively, in some embodiments, a conjugated polymer of the photo-thermal particles is a copolymer of two or more repeating units. For example, a conjugated polymer can be constructed of two or more monomeric species selected from the group consisting of D and A monomeric species described herein. In some embodiments, conjugated polymer is a copolymer of a donor-acceptor (D-A) architecture. For example, a D-A conjugated polymer can be composed of cyclopentadithiophene and 2,1,3-benzothiadiazole (PCPDTBT) or cyclopentadithiophene and 2,1,3-benzoselenadiazole (PCPDTBSe). In some embodiments, a conjugated polymer has the structure of Formula (I):

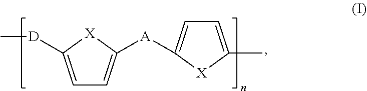

wherein D is a donor monomeric species described herein, A is an acceptor monomeric species described herein, and each X is independently O, N, S or Se. In some embodiments, a D-A conjugated polymer of the photo-thermal particles is of formula:

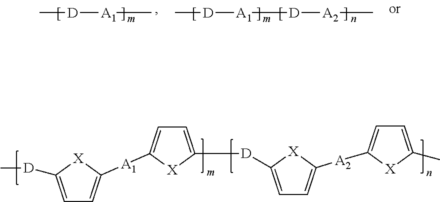

wherein D is a donor monomeric species described herein, A₁ and A₂ are acceptor monomeric species described herein. In some embodiments, m and n range from 1 to 100.

In some embodiments, conjugated polymeric species of the photo-thermal particles comprise polyethylenedioxythiophene (PEDOT). The photo-thermal particles, for example, can be formed of PEDOT or a polymeric mixture comprising PEDOT. In some embodiments, photo-thermal particles comprise PEDOT-polystyrene sulfonate (PSS).

Composition of the photo-thermal particles can be uniform or substantially uniform in a composite composition. Alternatively, photo-thermal particle composition can vary in the composite composition. Photo-thermal particles of differing composition can be dispersed in the polymeric matrix. In some embodiments, photo-thermal particles may be of the same conjugated polymer, but differing molecular weight and/or size. In this way, properties of composite polymeric compositions described herein can be tailored.

Conjugated polymeric species of the photo-thermal particles, in some embodiments, have sufficient molecular weight to red-shift the absorption profile of the conjugated polymeric species into the red region of the visible spectrum and/or into the near infrared region. In some embodiments, for example, a conjugated polymeric species of the photo-thermal particles has a number average molecular weight (Mₙ) of at least 7500. Molecular weight of a polymeric species in the photo-thermal nanoparticles, in some embodiments, is selected from Table I.

TABLE I
Conjugated Photo-thermal Polymer
Molecular Weight (Number Average)
5500-25000
6000-20000
7500-17000
9000-16000
10000-15000

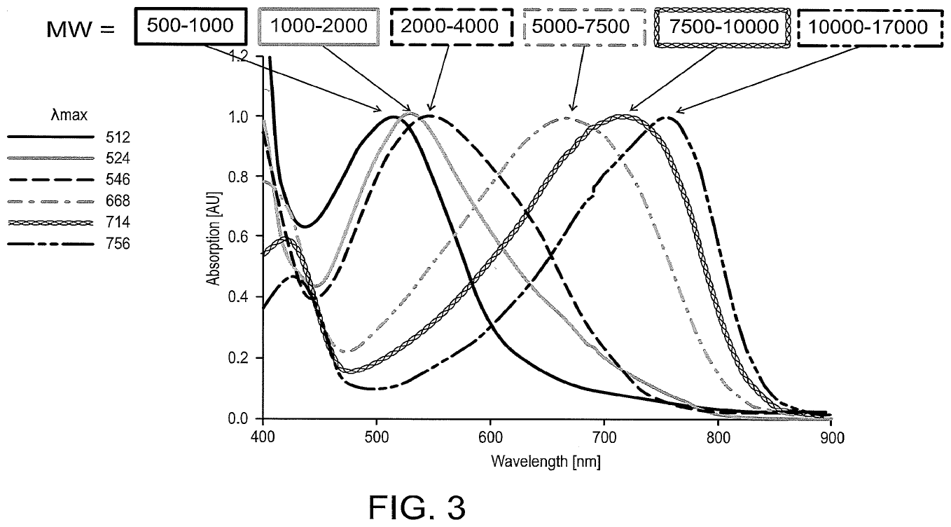

FIG. 3 illustrates absorption spectra red-shifting of PCPDTBSe with increasing number average molecular weight according to one embodiment described herein.

Photo-thermal particles of composite polymeric compositions described herein can have any desired size. Size of the photo-thermal particles can be selected according to several considerations including, but not limited to, compatibility of the particles with the polymeric matrix, concentration of the particles in the polymeric matrix, thermal responsiveness of the composite composition and/or mechanical properties of the composite composition. In some embodiments, photo-thermal particles are nanoparticles, microparticles or mixtures thereof. The photo-thermal particles can exhibit bimodal or multimodal particle size distributions.

In some embodiments, a photo-thermal nanoparticle has particle size in the range of 0.1 nm to 500 nm. A photo-thermal nanoparticle can have a size selected from Table II.

```
TABLE II
Photo-thermal Nanoparticle Size (nm)
  1-500
10-300
10-200
15-180
20-150
  5-100
```

As described herein, the photo-thermal particles are dispersed in a polymeric matrix. Any polymeric matrix not inconsistent with the objectives of the present invention can be employed. Polymeric matrices may be selected according to several considerations including, but not limited to, spectral characteristics, thermal conductivity, desired mechanical properties and/or dispersibility of the photo-thermal particles in the matrix. In some embodiments, the polymeric matrix can be transparent to visible radiation. Alternatively, the polymeric matrix can be colored or opaque. Suitable polymeric matrices can be formed of thermoplastics, thermosets and/or elastomers. For example, polymeric matrix can be selected from the group consisting rubber, silicone elastomer, siloxanes, polyisoprene and polyisobutylene. Photo-thermal particles can be present in the polymeric matrix in any desired amount or concentration. In some embodiments, photo-thermal particles are present in the polymeric matrix at a concentration of 0.1 mg/ml to 100 mg/ml. Photo-thermal particles can also be present in the polymeric matrix at a concentration selected from Table III.

TABLE III
Photo-thermal Particle Concentration (mg/ml)
0.5-50
0.75-30
1-25
2-15
5-10
0.5-15

In some embodiments, photo-thermal particles are present in an amount or concentration sufficient for the destruction of microbes and associated biofilms. As shown in the examples detailed herein, the composite polymeric compositions are effective in killing bacteria and associated bacterial biofilms. In some embodiments, photo-thermal particles of composite polymeric compositions described herein can have composition and properties described in U.S. patent application Ser. Nos. 14/394,198 and 15/303,171 which are incorporated herein by reference in their entireties. Photo-thermal particles, for example, can exhibit a hybrid architecture comprising a low molecular weight conjugated polymer component and a high molecular weight conjugated polymer component. The low and high molecular weight conjugated polymers may be of the same or different composition.

Photo-thermal particles can be combined or dispersed in the polymeric matrix in any desired manner. In some embodiments, photo-thermal particles are physically mixed with the polymeric matrix. In other embodiments, the photo-thermal particles are present during polymerization and/or cross-linking of the polymeric matrix. Photo-thermal particles, for example, can be added to the polymerization reaction mixture.

In another aspect, articles employing the composite polymeric compositions are provided. In some embodiments, an article comprises one or more surfaces and a coating over the one or more surfaces, the coating comprising a polymeric matrix and photo-thermal particles comprising conjugated polymer dispersed in the polymeric matrix. The coating can have any desired thickness. Coating thickness can be selected according to several considerations including, but not limited to, light absorption and/or heating characteristics of the coating, wettability of the coating on article surfaces and adhesion of the coating to article surfaces. In some embodiments, the coating is a single layer. In other embodiments, the coating is multilayer. In multilayer embodiments, individual layers can have the same or differing compositions and/or properties. Individual coating layers, for example, can have the same or substantially the same thickness and/or composition. In other embodiments, individual coating layers can have differing thicknesses and/or composition. Specific coating architecture can be tailored to the desired spectral absorption and associated heating requirements of the coating.

In some embodiments, a layer having good compatibility with article surfaces can serve as the base layer of the coating for enhancing coating adhesion. The base layer may or may not have desirable absorbance or hearting characteristics. However, subsequent layers of the coating can mitigate any performance deficiencies of the base layer. Surfaces of the article can also be prepared to enhance coating adhesion. In some embodiments, article surfaces can be mechanically treated to enhance coating adhesion. Mechanical treatment can include roughing or smoothing article surfaces. Article surfaces may also be chemically treated prior to applications of coatings described herein. Chemical treatment can alter surface energies, thereby enhancing compatibility with the coating. One or more adhesion layers may also be applied to article surfaces. In some embodiments, the adhesion layers do not comprise photo-thermal particles.

Any article not inconsistent with the objectives of the present invention can have surfaces coated with the composite polymeric composition. As described further herein, articles supporting high concentrations of microbes and associated biofilms can be coated with composite polymeric compositions described herein. Various hospital surfaces, medical devices and/or implants can be coated with composite polymeric compositions described herein. In further embodiments, articles can be formed of composite polymeric compositions described herein. In such embodiments, the articles can be employed as heat sources for enhancing uptake and/or efficacy of various pharmaceutical compositions including antimicrobial or antibacterial compositions and/or chemotherapeutic compositions.

In a further aspect, methods of cleaning surfaces are provided. A method of cleaning a surface comprises heating the surface via exposure to electromagnetic radiation to kill and/or disperse microbes, the surface comprising a composite polymeric composition including a polymeric matrix and photo-thermal nanoparticles comprising conjugated polymer dispersed in the polymeric matrix.

These and other embodiments are further illustrated in the following non-limiting examples.

### Example 1—Synthesis of PCPDTBT and PCPDTBSe

All reagents were purchased from common commercial sources and used without further purification unless otherwise noted. 4H-Cyclopenta-[1,2-b:5,4-b]dithiophene was purchased from Astar Pharma. THF was dried over Na/benzophenone ketal. 4,4-Bis(2-ethylhexyl)-2,6-bis(trimethylstannyl)-4H-cyclopenta-[2,1-b;3,4-b′]dithiophene, 4,7-dibromo-2,1,3-benzothiadiazole and 4,7-dibromo-2,1,3-benzoselenadiazole were synthesized according to their literature procedures (see J. Hou, T. L. Chen, S. Zhang, H.-Y. Chen, Y. Yang, J. Phys. Chem. C 2009, 113, 1601-1607; Z. Zhu, D. Waller, R. Gaudiana, M. Morana, D. Muhlbacher, M. Scharber, C. Brabec, Macromolecules 2007, 40, 1981-1986; C. W. Bird, G. W. H. Cheeseman, A. A. Sarsfield, J. Chem. Soc. 1963, 4767-4770; I. H. Jung, H. Kim, M.-J. Park, B. Kim, J.-H. Park, E. Jeong, H. Y. Woo, S. Yoo, H.-K. Shim, J. Polym. Sci. Part A: Polym. Chem. 2010, 48, 1423-1432; X. Li, W. Zeng, Y. Zhang, Q. Hou, W. Yang, Y. Cao, Eur. Polym. J. 2005, 41, 2923-2933; and Y. Tsubata, T. Suzuki, T. Miyashi, Y. Yamashita, J. Org. Chem. 1992, 57, 6749-6755, the entireties of which are hereby incorporated by reference). Poly[4,4-bis(2-ethylhexyl)-cyclopenta[2,1-b;3,4-b′]dithiophene-2,6-diylalt-2,1,3-benzothiadiazole-4,7-diyl] (PCPDTBT) and poly[4,4-bis(2-ethylhexyl)-cyclopenta[2,1-b;3,4-b′]dithiophene-2,6-diylalt-2,1,3-benzoselenadiazole-4,7-diyl] (PCPDTBSe) were synthesized using a Stille coupling procedure under microwave radiation. The polymerization procedure is outlined below.

Flash chromatography was performed on a Biotage Isolera™ Flash Purification System using Biotage SNAP Flash Purification Cartridges as the stationary phase. Microwave assisted polymerizations were carried out using a CEM Discover Microwave reactor. 300 and 500 MHz ¹H-NMR spectra were recorded on Bruker Avance DPX-300 and DRX-500 Instruments, respectively. ¹³C NMR spectra were recorded on a Bruker Avance DRX-500 instrument at 125.76 MHz. UV-Vis absorption spectra were recorded on an Agilent 8453 diode-array spectrophotometer operating over a range of 190-1100 nm. GC-MS were recorded on an Agilent 6850 Series GC system coupled to an Agilent 5973 mass selective detector run in electron impact mode. Infrared spectra were recorded either on a Mattson Genesis II FT-IR spectrometer or on a Perking-Elmer Spectrum 10 spectrometer with an ATR sampling accessory equipped with a diamond anvil Raman spectra were recorded on a DeltaNu Advantage 532 Raman spectrometer at 532 nm.

### Synthesis of PCPDTBT.

4,4-Bis(2-ethylhexyl)-2,6-bis(trimethylstannyl)-4H-cyclopenta-[2,1-b;3,4-b′]dithiophene was added to a microwave tube along with 4,7-dibromo-2,1,3-benzothiadiazole (1.05:1 equivalent) and 2 mL of chlorobenzene. The tube was stirred for 5 minutes to dissolve the monomers. Pd(PPh₃)₄ (2.5 mol %) was then added and the tube was sealed with a crimp cap and placed in a microwave reactor where it was heated to 200° C. for 10 minutes. Upon cooling to room temperature a viscous solution of green polymer was observed in the reaction vessel. The polymer was precipitated in methanol and collected by vacuum filtration. The solid was then transferred to a Soxhlet thimble and subjected to extraction with MeOH (3 hrs), hexanes (6 hrs), and finally chloroform (6 hrs). The chloroform extract was evaporated almost to completion and methanol was added to precipitate the polymer, which was filtered and dried under vacuum for 24 hours. ¹H-NMR is comparable to the literature values.

### Synthesis of PCPDTBSe.

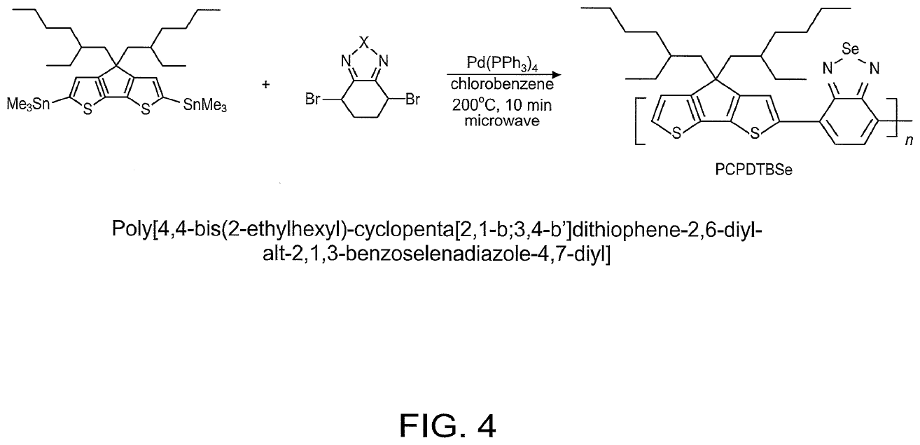

The synthesis of PCPDTBSe follows the same procedure as PCPDTBT above, except 4,7-dibromo-2,1,3-benzoselenadiazole (1.05:1 equivalent) was used instead of 4,7-dibromo-2,1,3-benzothiadiazole. ¹H-NMR was comparable to the literature values. FIG. 4 illustrates a synthetic pathway for PCPDTBSE.

### Example 2—Synthesis of PCPDTBSe Photo-Thermal Nanoparticles of Hybrid Architecture

1 mL of low molecular weight PCPDTBSe [2.15 mg/mL in THF] and 1 mL of high molecular weight PCPDTBSe [1 mg/mL in THF] were premixed and injected under continuous horn sonication (10% amplitude, 1 minute) into 8 mL of Pluronic F127 [5 mg/mL in water]. This solution was centrifuged (30 minutes 12,600 Gs) to pellet large nanoparticles; the resulting supernatant was then centrifuged (4 hours 12,600 Gs) to pellet small nanoparticles.

### Example 3—Synthesis of PCPDTBSe Photo-Thermal Nanoparticles of Core-Shell Architecture

1 mL of high molecular weight PCPDTBSe [1 mg/mL in THF] followed by 1 mL of low molecular weight PCPDTBSe [2.15 mg/mL in THF] was injected under continuous horn sonication (10% amplitude, 1 minute) into 8 mL of Pluronic F127 [5 mg/mL in water]. This solution was centrifuged (30 minutes 12,600 Gs) to pellet large nanoparticles; the resulting supernatant was then centrifuged (4 hours 12,600 Gs) to pellet small nanoparticles.

PCPDTBSe photo-thermal nanoparticles described herein were combined with elastomeric matrix to produce thermally-active polymeric composites detailed in Appendix A.

### Example 4—Synthesis of Composite Polymeric Material

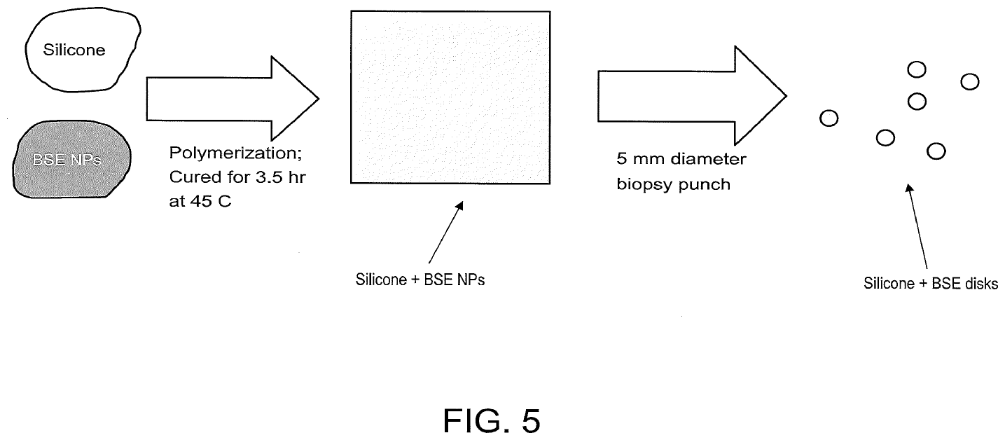

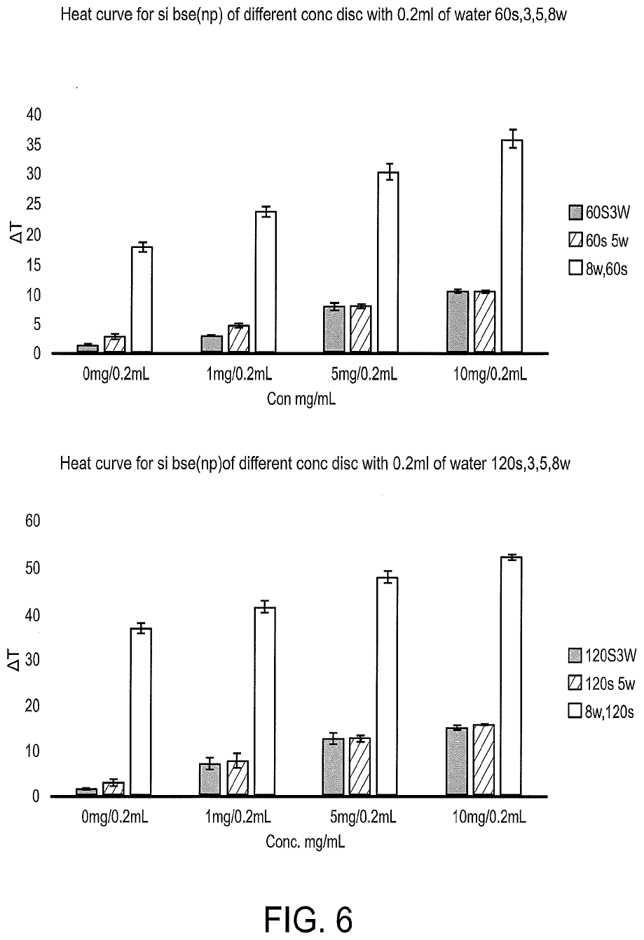

PCPDTBSe (“BSE”) nanoparticles were synthesized in accordance with Example 1 herein. The BSE nanoparticles were combined with a silicone matrix as set forth in FIG. 5 to provide a polymeric composite material. BSE-silicone composite materials were synthesized with BSE nanoparticle loadings of 1 mg/ml, 5 mg/ml and 10 mg/ml. The sheets of polymeric composite were subsequently cut into discs having diameter of 5 mm. The heating profiles of the composite polymeric compositions in 0.2 ml of water were then investigated. Laser powers of 3 W, 5 W and 8 W in the near infrared (NIR) were employed along with irradiation times of 60 seconds and 120 seconds. The resulting heating profiles of the Si-BSE composites are provided in FIG. 6.

### Example 5—Ablation of Bacterial Species

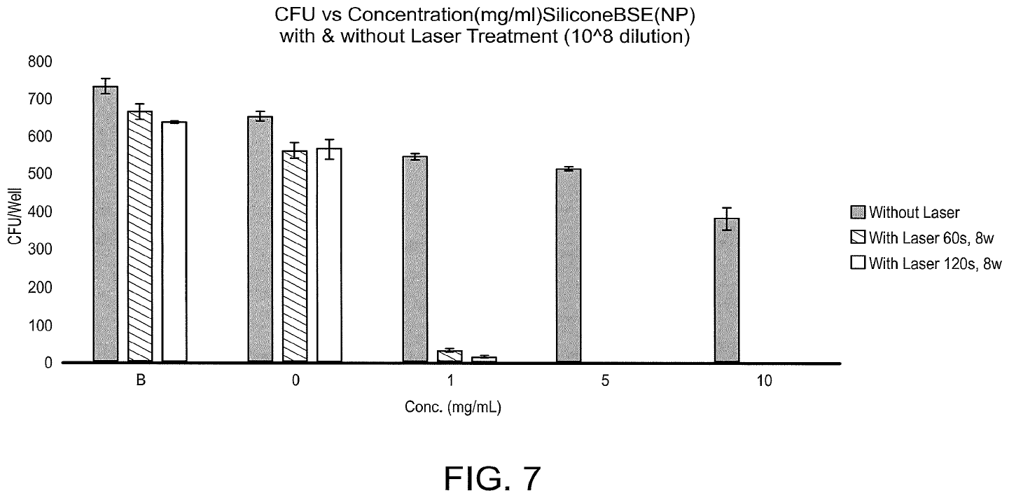

The Si-BSE composites of Example 4 were tested for ablation efficacy of microbial species. In particular, the Si-BSE composites were combined with planktonic S. Aureus and subjected to near infrared (NIR), 8 W laser treatment at 60 s and 120 s. The results of the testing are provided in FIG. 7. As illustrated in FIG. 7, irradiation of the BSE-Si composites achieved sufficient hearting for ablation of the S. Aureus.

### Example 6—Antimicrobial Activity of Polymeric Composites

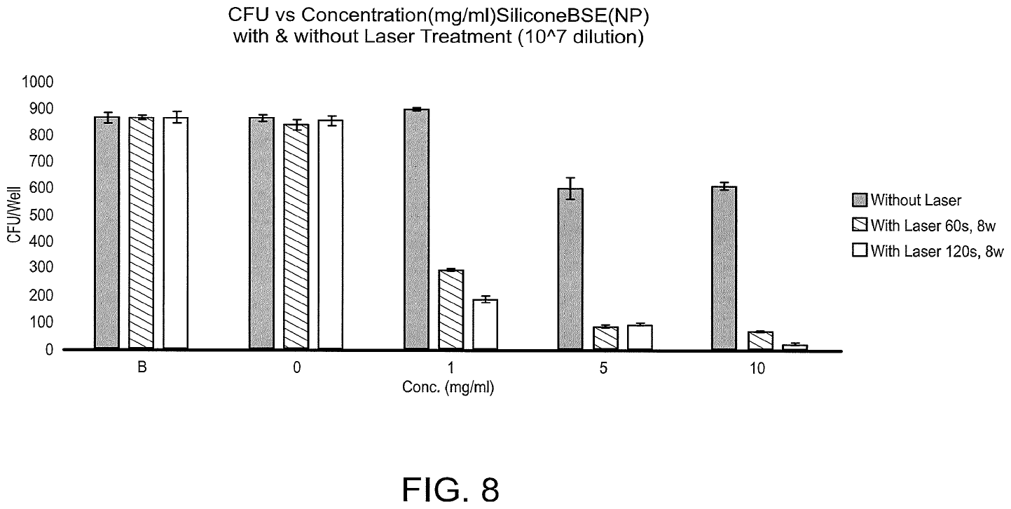

The antimicrobial activity of Si-BSE composites of Example 4 were tested against S. Aureus biofilms with and without laser irradiation. Two near infrared (NIR) laser irradiation conditions were employed: (1) 8 W for 60 seconds and (2) 8 W for 120 seconds. The results of the testing are provided in FIG. 8. Onset of significant bacterial death occurred at BSE concentration of 1 mg/ml under laser treatment. Notably, significant bacterial death was also observed without laser treatment for the BSE concentrations of 5 mg/ml and 10 mg/ml. Accordingly, the polymeric composites are operable to exhibit antimicrobial behavior in the absence of laser treatment.

### Example 7—Antimicrobial Activity of Polymeric Composites

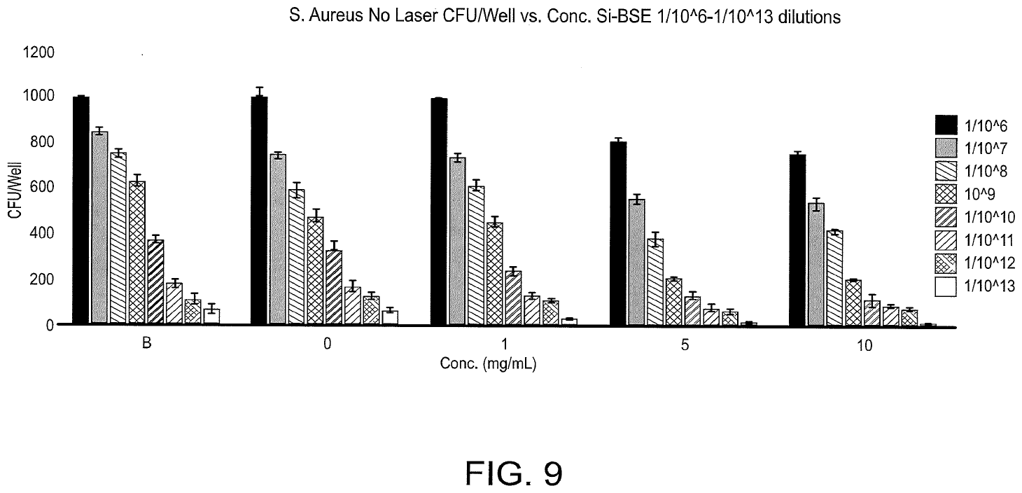

The antimicrobial activity of Si-BSE composites of Example 4 were tested against S. Aureus biofilms without laser irradiation. The results are provided in FIG. 9. In the absence of laser treatment, the Si-BSE composites were able to reduce the number of cells of S. Aureus at 10¹³ dilution. The antimicrobial activity of the Si-BSE composites in the absence of laser irradiation may further the efficacy of the composite for resisting biofilm formation.

### Example 8—Antimicrobial Activity of Polymeric Composites

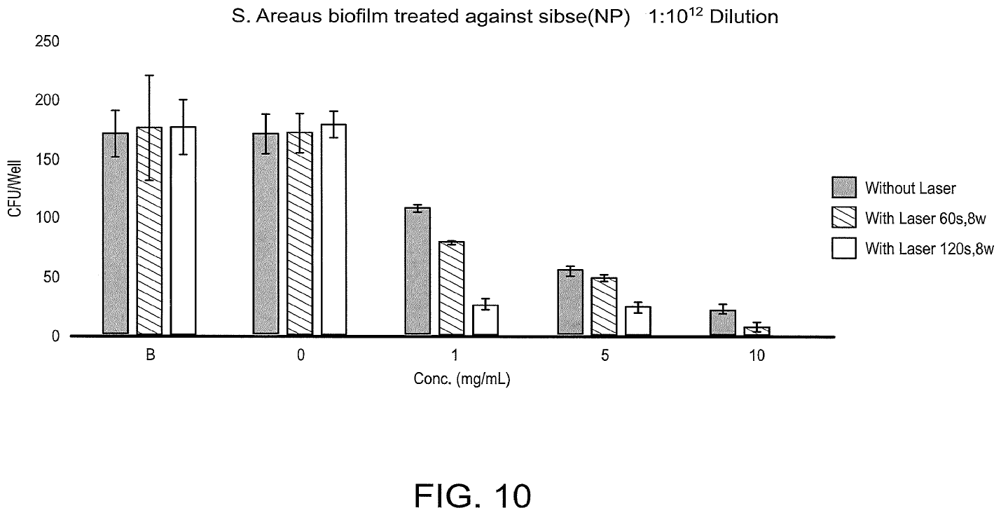

The antimicrobial activity of Si-BSE composites of Example 4 were tested against S. Aureus biofilms with and without laser irradiation. Two near infrared (NIR) laser irradiation conditions were employed: (1) 8 W for 60 seconds and (2) 8 W for 120 seconds. The results of the testing are provided in FIG. 10. As illustrated in FIG. 10, laser irradiation killed nearly all of the bacteria. Moreover, significant antimicrobial activity of the Si-BSE composites was evidenced in the absence of laser irradiation.

### Example 9—Polymeric Composites in Conjunction with Antibiotic Compound

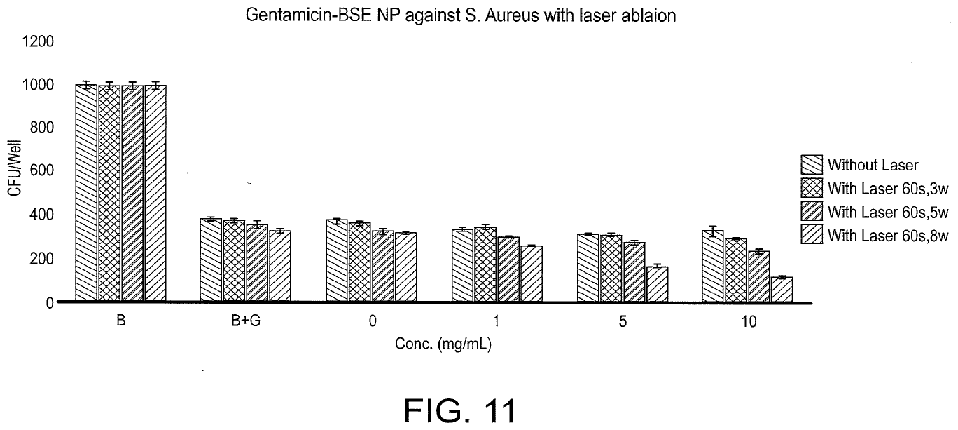

Si-BSE composites of Example 4 were combined with the antibiotic Gentamicin and tested against S. Aureus under varying NIR laser treatment conditions. The NIR laser treatment conditions were 3 W for 60 seconds, 5 W for 60 seconds and 8 W for 60 seconds. The results are provided in FIG. 11. Combination of Gentamicin with the Si-BSE composites followed by laser treatment lead to significant reduction in bacterial colonies.

### Example 10—Polymeric Composites in Conjunction with Antibiotic Compound

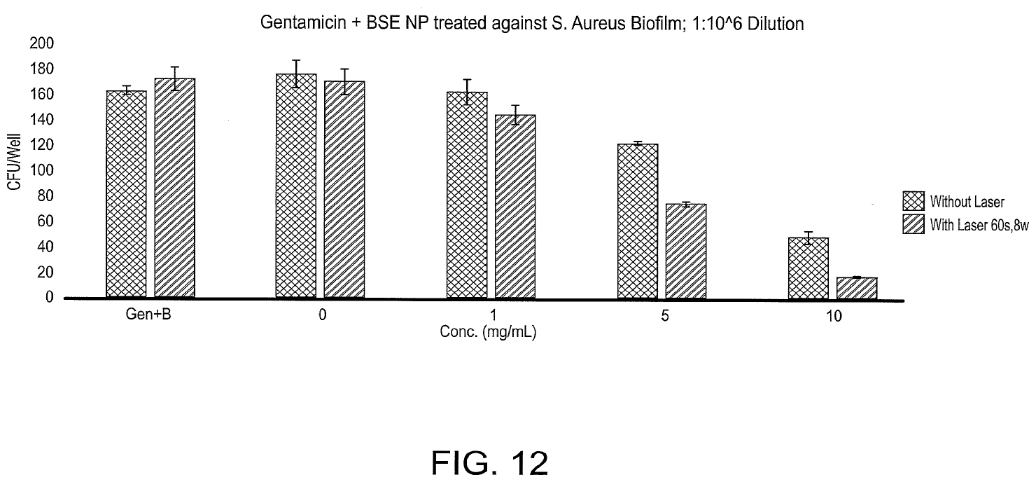

Si-BSE composites of Example 4 were combined with the antibiotic Gentamicin and tested against S. Aureus biofilm under NIR laser treatment at 8 W and 60 seconds. The results are provided in FIG. 12. As illustrated in FIG. 12, significant biofilm reduction was observed for higher BSE concentrations in the polymeric composite in conjunction with Gentamicin.

### Example 11—Articles Coated with Polymeric Composites

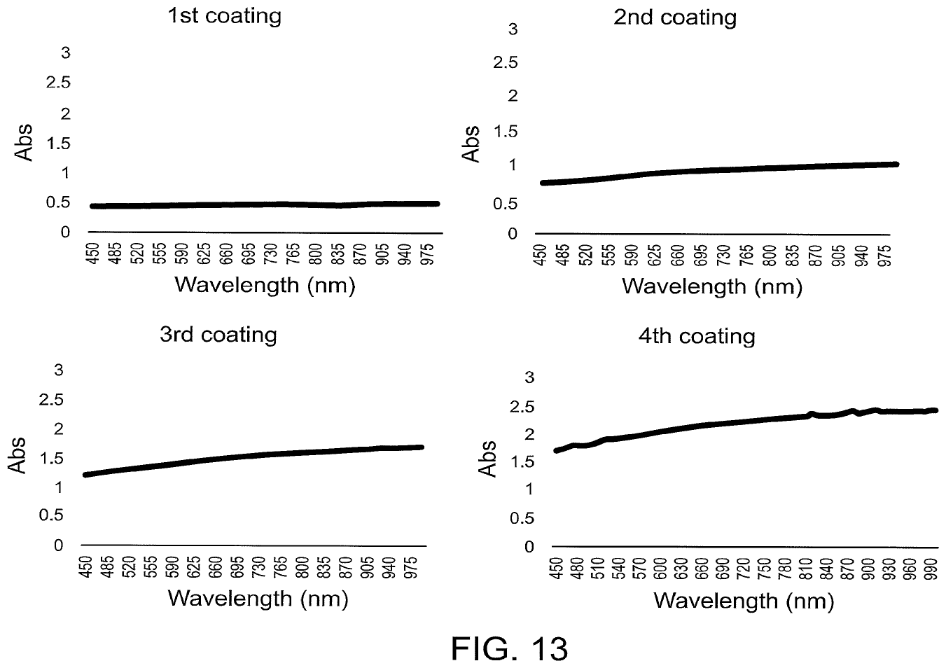

5 mg of polyethylenedioxythiophene (PEDOT) nanotubes and 1 g of silicone base (Sylgard 184) were ground using a pestile. After completion of the grinding, 3 drops (0.01 g) of Sylgard 184 crosslinker were added to the mixture. Two pieces of VINCON® tubing (12 cm diameter) were coated with the resulting mixture and placed in an oven at 50° C. for curing. This process was repeated up to 4 times to provide tubing comprising 4 layers of the polymeric composite. The samples were subsequently tested for light absorbance. The results are provided in FIG. 13. Each sample exhibited a maximum absorbance of about 970 nm, with the 4-layer coating exhibiting the greatest absorbance.

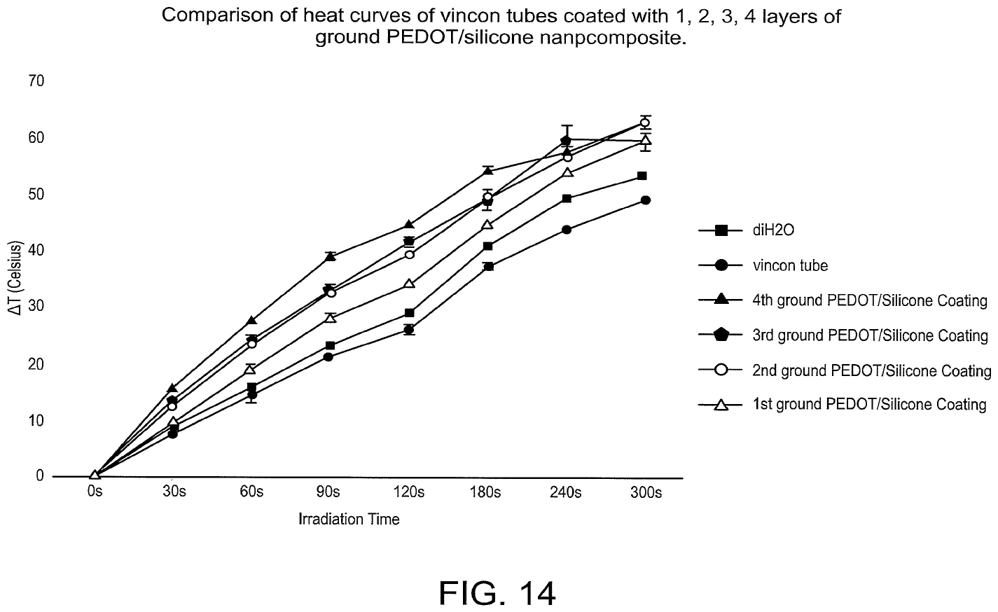

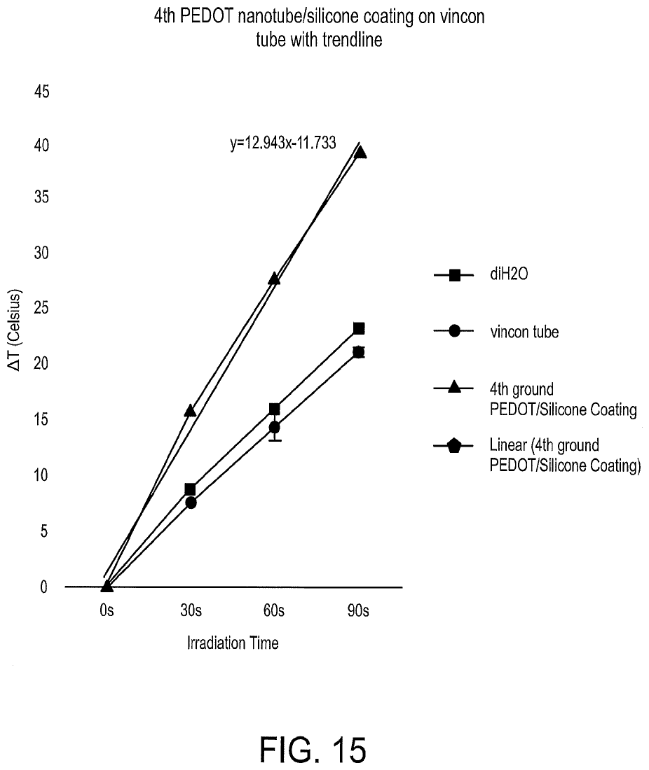

A 1 cm piece was cut from each sample and inserted into a well of a 24-well plate filled with 2 mL of water. A continuous wave, 5 W, 970 nm laser was used to irradiate the samples for various time periods, and the temperature change in the water was measured. Irradiation times were 30 seconds, 60 seconds, 90 seconds, 120 seconds, 180 seconds, 240 second and 300 seconds. All samples were conducted in triplicate, and a 1 cm piece of uncoated VINCON® in 2 ml of water was used as the control. FIG. 14 provides the results of the laser testing. As illustrated in FIG. 14, the samples provided rapid heating of the water in response to laser radiation. The VINCON® tubing comprising the 4-layer coating exhibited high heating potential and structural stability. FIG. 15 illustrates additional heating data for this 4-layer sample.

### Example 12—Articles Coated with Polymeric Composites

5 mg of polyethylenedioxythiophene (PEDOT) nanotubes and 1 g of silicone base (Sylgard 184) were mixed with a spatula. After completion of the mixing, 3 drops (0.01 g) of Sylgard 184 crosslinker were added to the mixture. Two pieces of VINCON® tubing (12 cm diameter) were coated with the mixture and placed in an oven at 50° C. for curing. This process was repeated up to 4 times to provide tubing comprising 4 layers of the polymeric composite.

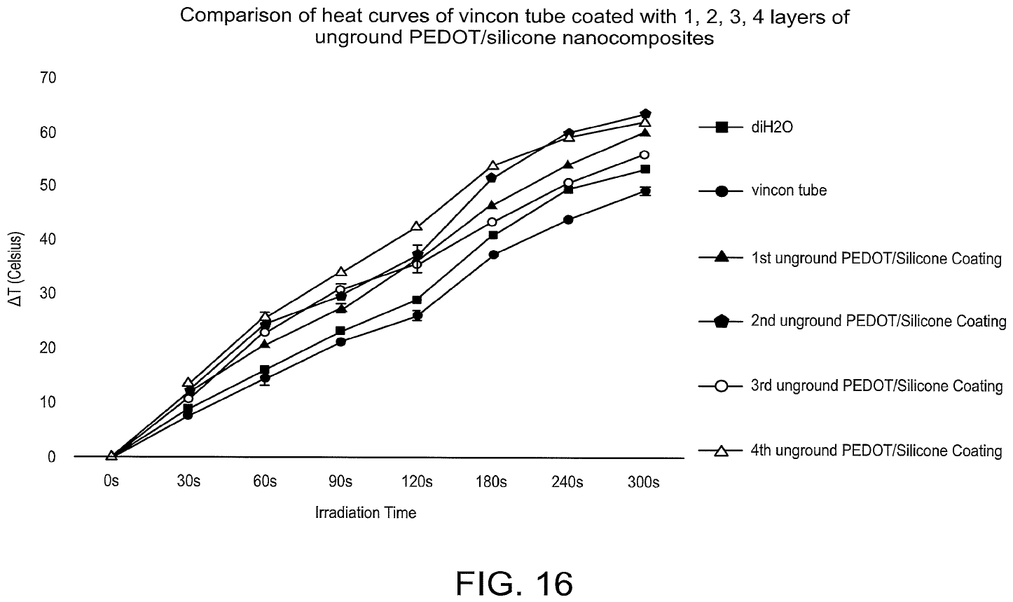

A 1 cm piece was cut from each sample and inserted into a well of a 24-well plate filled with 2 mL of water. A continuous wave, 5 W, 970 nm laser was used to irradiate the samples for various time periods, and the temperature change in the water was measures. Irradiation times were 30 seconds, 60 seconds, 90 seconds, 120 seconds, 180 seconds, 240 second and 300 seconds. All samples were conducted in triplicate, and a 1 cm piece of uncoated VINCON® in 2 ml of water was used as the control. FIG. 16 provides the results of the laser testing. As illustrated in FIG. 16, the samples provided rapid heating of the water in response to laser radiation. Moreover, the heating profiles of these unground PEDOT samples were similar to the ground PEDOT samples of Example 11.

### Example 13—Polymeric Composite Heating Profiles

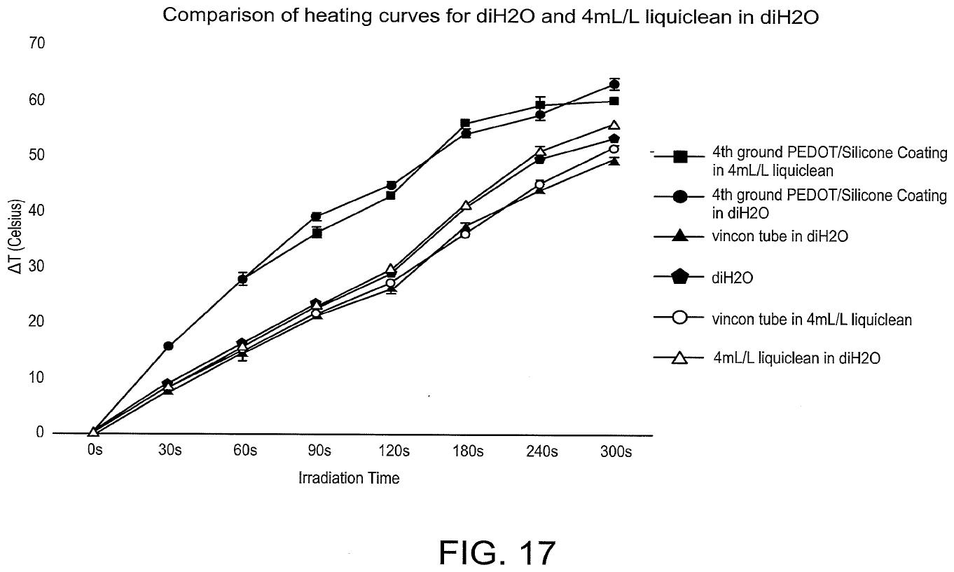

The heating profiles of the 4-layer samples of Examples 11 and 12 were examined with respect to LIQUICLEAN® detergent, commercially available from Ruholf Corporation of Mineola, N.Y. The same laser treatment of Examples 11 and 12 was applied in the present example. The results are provided in FIG. 17. As illustrated in FIG. 17, the heating profiles in detergent did not substantially differ from the heating profiles in deionized water.

Various embodiments of the invention have been described in fulfillment of the various objectives of the invention. It should be recognized that these embodiments are merely illustrative of the principles of the present invention. Numerous modifications and adaptations thereof will be readily apparent to those skilled in the art without departing from the spirit and scope of the invention.

1. A composite polymeric composition comprising:
a polymeric matrix; and
photo-thermal particles comprising conjugated polymer dispersed in the polymeric matrix.

2. The composite polymeric composition of claim 1, wherein the photo-thermal particles have an average size of 1 nm to 500 nm.

3. The composite polymeric composite of claim 1, wherein the photo-thermal particles are present in the composition at a concentration of 0.1-100 mg/ml.

4. The composite polymeric composite of claim 1, wherein the photo-thermal particles are present in the composition at a concentration of 0.5-15 mg/ml.

5. The composite polymeric composite of claim 1, wherein the conjugated polymer has a bandgap of 1.1 eV to 1.8 eV.

6. The composite polymeric composite of claim 5, wherein the conjugated polymer is a homopolymer.

7. The composite polymeric composition of claim 1, wherein the conjugated polymer has a donor-acceptor architecture comprising a donor monomeric species (D) and an acceptor monomeric species (A).

8. The composite polymeric composition of claim 7, wherein the acceptor monomeric species is selected from the group consisting of a monocyclic arylene, bicyclic arylene and polycyclic arylene.

9. The composite polymeric composition of claim 7, wherein the donor monomeric species is a substituted or unsubstituted fused dithiophene and the acceptor monomeric species is a substituted or unsubstituted benzodiazole.

10. The composite polymeric composition of claim 9, wherein the fused dithiophene is of the formula 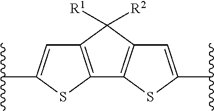
and the benzodiazole is of the formula 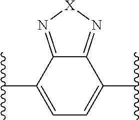
wherein R¹ and R² are selected from the group consisting of hydrogen, alkyl, alkenyl, aryl, heteroaryl, O-alkyl, O-alkenyl, and O-aryl and wherein X is selected from the group consisting of oxygen, nitrogen, sulfur and selenium.

11. The composite polymeric composition of claim 1, wherein the polymeric matrix comprises thermoplastic, thermoset or elastomer.

12. The composite polymeric composition of claim 11, wherein the polymeric matrix is transparent to visible radiation.

13. The composite polymeric composition of claim 11, wherein the polymeric matrix is the elastomer and the photo-thermal particles are encapsulated in the elastomer.

14. The composite polymeric composition of claim 1, wherein the photo-thermal particles comprise conjugated polymers of differing molecular weight.

15. The composite polymeric composition of claim 1, wherein two or more of the photo-thermal particles have differing composition.

16. The composite polymeric composition of claim 1, wherein two or more of the photo-thermal particles have differing electromagnetic radiation absorption profiles.

17. An article comprising;
one or more surfaces; and
a coating over the one or more surfaces, the coating comprising a polymeric matrix and photo-thermal nanoparticles comprising conjugated polymer dispersed in the polymeric matrix.

18. The article of claim 17, wherein the photo-thermal particles have an average size of 1 nm to 500 nm.

19. The article of claim 17, wherein the photo-thermal particles are present in the coating at a concentration of 0.1-100 mg/ml.

20. The article of claim 17, wherein the coating is a single layer coating.

21. The article of claim 17, wherein the coating is a multilayer coating.

22. The article of claim 21, wherein two or more individual layers of the multilayer coating differ in at least one property.

23. The article of claim 22, wherein the two or more individual layers differ in electromagnetic radiation absorption profile.

24. The article of claim 22, wherein two or more individual layers differ in photo-thermal particle loading.

25. The article of claim 17, wherein the coating directly contacts the article surface.

26. The article of claim 17, wherein the polymeric matrix comprises thermoplastic, thermoset or elastomer.

27. The article of claim 17, wherein the polymeric matrix is transparent to visible radiation.

28. The article of claim 17, wherein the photo-thermal particles are encapsulated by the polymeric matrix.

29-33. (canceled)
